(19) United States
(12) Patent Application Publication
(10) Pub. No.: US 2017/0325849 A1
(43) Pub. Date: Nov. 16, 2017

(54) MULTIPLE DIAMETER SPINAL ROD CLAMPING MECHANISM

(71) Applicant: Spineology Inc., St. Paul, MN (US)

(72) Inventor: Joseph Gleason, Eagan, MN (US)

(21) Appl. No.: 15/594,586

(22) Filed: May 13, 2017

Related U.S. Application Data
(60) Provisional application No. 62/336,554, filed on May 13, 2016.

(51) Int. Cl. A61B 17/70; A61B 17/56

(52) U.S. Cl. CPC A61B 17/7052; A61B 2017/564

(57) ABSTRACT

A cross-connector for spinal rods includes an adjustable rod clamping mechanism that adjusts to a variety of rod diameters to be clamped on each side of the cross-connector. The adjustable rod clamping mechanism includes a fixed hook portion and a cross-slide cam driven by a tapered set screw that is oriented 90 degrees to the travel of the cam.

### PRIORITY

This application claims the benefit of U.S. Provisional Application Ser. No. 62/336,554, filed on May 13, 2016, which is hereby incorporated herein by reference in its entirety.

### FIELD

The present invention generally relates to devices used in spinal surgeries, and more particularly to cross-connectors used in spinal screw/rod constructs.

### BACKGROUND

In spinal surgical procedures, cross-connectors are used in spinal screw/rod constructs. In such constructs, screws are placed on either side of the spine with interconnecting rods which generally run parallel to each other. Cross-connectors are used to span between the parallel rods to further box connect the parallel rods for additional rigidity.

Conventional cross-connectors have to be selected based upon the diameter of the rods to which the connectors will be secured. This requires that the surgeon use only a rod diameter for which they have a correspondingly-sized cross-connector. This also requires that the surgeon have available a variety of differently-configured cross-connectors so that desired rod diameters can be employed in a given construct.

Thus, there is a need for an improved cross-connector that can adjust to a variety of rod diameters.

### SUMMARY

The disclosure includes an adjustable rod clamping mechanism for a cross-connector to adjust to the diameters of the rods to be clamped on each side of the cross-connector. The disclosure also includes a cross-connector featuring an adjustable rod clamping mechanism that adjusts to the diameters of the rods to be clamped on each side of the cross-connector. The disclosure further includes a method of adjusting a cross-connector clamping mechanism to secure a range of rod diameters. The disclosure additionally includes a system to adjust a cross-connector clamping mechanism to secure a range of rod diameters.

In one of the disclosed examples, a cross-connector includes an adjustable rod clamping mechanism that adjusts to the diameters of the rods to be clamped on each side of the cross-connector. The adjustable rod clamping mechanism includes a fixed hook portion and a cross slide cam driven by a tapered set screw that is oriented 90 degrees to the travel of the cam. The fixed hook and the cross slide cam include portions corresponding to each of two or more different diameters contoured into them.

The disclosure includes an adjustable spinal rod cross-connector. The cross-connector includes a bridge member, a first clamping mechanism disposed on a first end of the bridge member, and a second clamping mechanism disposed on an opposing second end of the bridge member. The first and second clamping mechanisms each include a cross-slide cam, including a forward surface defining a curved portion and a rear surface defining an angled plane with respect to a horizontal plane, and a set screw movable in a direction perpendicular to a long axis of the cross-slide cam and located such that the set screw engages the angled plane of the rear surface of the cross-slide cam.

The set screw can include a beveled tip with a bevel angle defined with respect to the horizontal plane. The bevel angle can be greater than an angle defined between the angled plane and the horizontal plane. In one example the bevel angle can be sixty degrees and the angled plane can form a thirty degree angle with respect to the horizontal plane.

Each of the first and second clamping mechanisms can include a fixed hook portion that defines a curved inner surface opposite the curved portion of the cross-slide cam.

The cross-slide cam can have sufficient travel between a fully extended position and a fully retracted position to engage spinal rods having diameters ranging from 4.5 mm to 6.0 mm. The cross-slide cam can also have sufficient travel between a fully extended position and a fully retracted position to engage spinal rods having diameters of both 4.75 mm and 5.50 mm.

The disclosure also includes an adjustable clamping mechanism for a spinal rod cross-connector. The adjustable clamping mechanism includes a clamp body defining a fixed hook portion. The fixed hook portion defines an inner surface. A cross-slide cam is disposed in the clamp body. The cross-slide cam includes a forward surface defining a curved portion and a rear surface defining an angled plane. The cross-slide cam is movable along a long axis thereof through the body so that the forward surface moves towards and away from the curved inner surface of the fixed hook portion. A set screw is disposed in the body and oriented 90 degrees to the long axis of the cross-slide cam such that the set screw engages the angled plane of the rear surface of the cross-slide cam.

The set screw can define a beveled tip with a bevel angle defined with respect to the horizontal plane. The beveled angle can be greater than the angle defined between the angled plane of the rear surface of the cross-slide cam and the horizontal plane. For example, the bevel angle can be sixty degrees and the angled plane of the rear surface of the cross-slide cam can form a thirty degree angle with respect to the horizontal plane.

The cross-slide cam in the adjustable clamping mechanism can define sufficient travel between a fully extended position and a fully retracted position to engage spinal rods having diameters ranging from 4.5 mm to 6.0 mm.

The disclosure further includes a method of securing a spinal rod with a clamping mechanism. The method includes disposing a spinal rod in an adjustable clamping mechanism between a forward end of a cross-slide cam and a curved inner surface of a fixed hook portion of the clamping mechanism. A screw is turned in order to contract or move the screw into the adjustable clamping mechanism to move the cross-slide cam in a direction perpendicular to a direction of contraction of the set screw. A curved forward end of the cross-slide cam is engaged with the spinal rod to secure the spinal rod in the adjustable clamping mechanism between the curved forward end and the curved inner surface of the fixed hook portion.

An angled rear surface of the cross-slide cam can be deflected by a beveled tip of the screw. An angle of the beveled tip with respect to a horizontal plane can be greater than an angle of the angled rear surface of the cross-slide cam with respect to the horizontal plane. A fully extended position of the cross-slide cam and a fully retracted position of the cross-slide cam can each be defined to permit engagement with spinal rods having diameters of both 4.75 mm and 5.50 mm.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective view of a cross-connector according to certain embodiments.

FIG. 2 is a perspective view of a cross-connector according to certain embodiments.

FIG. 3 is a perspective view of a cross-connector according to certain embodiments.

FIG. 4 is a perspective view of a cross-connector according to certain embodiments.

FIG. 5 is a perspective view of a cross-connector according to certain embodiments.

FIG. 6 is a side view showing the adjustable clamping mechanism of a cross-connector according to certain embodiments wherein the cam is in a retracted position.

FIG. 7 is a cross-sectional view of the adjustable clamping mechanism of a cross-connector shown in FIG. 6.

FIG. 8 is a side view showing the adjustable clamping mechanism of a cross-connector according to certain embodiments wherein the cam is engaged with a larger of two rod diameters.

FIG. 9 is a cross-sectional view of the adjustable clamping mechanism of a cross-connector shown in FIG. 8.

FIG. 10 a side view showing the adjustable clamping mechanism of a cross-connector according to certain embodiments wherein the cam is engaged with a smaller of two rod diameters.

FIG. 11 is a cross-sectional view of the adjustable clamping mechanism of a cross-connector shown in FIG. 10.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention.

Spinal screw/rod constructs use rods commonly sized at 5.5 mm and 4.75 mm diameters. The present invention allows rigid connection to both diameters, which allows one cross-connector 100 to be applied to a first rod having a 5.5 mm diameter and a second rod having a 4.75 mm diameter. Additionally, both clamping mechanisms 102a and 102b of the cross-connector 100 can rigidly connect to spinal rods having the same diameter, but the same cross-connector 100 device can be used for both rod sizes, thus reducing inventory needs.

It is also included that the cross-connector 100 can be adapted to clamp other rod sizes (or ranges of sizes) than mentioned in the preceding paragraph. For example, the clamping mechanisms 102a, 102b can be adapted to also secure 6.0 mm diameter rods and 4.5 mm rods.

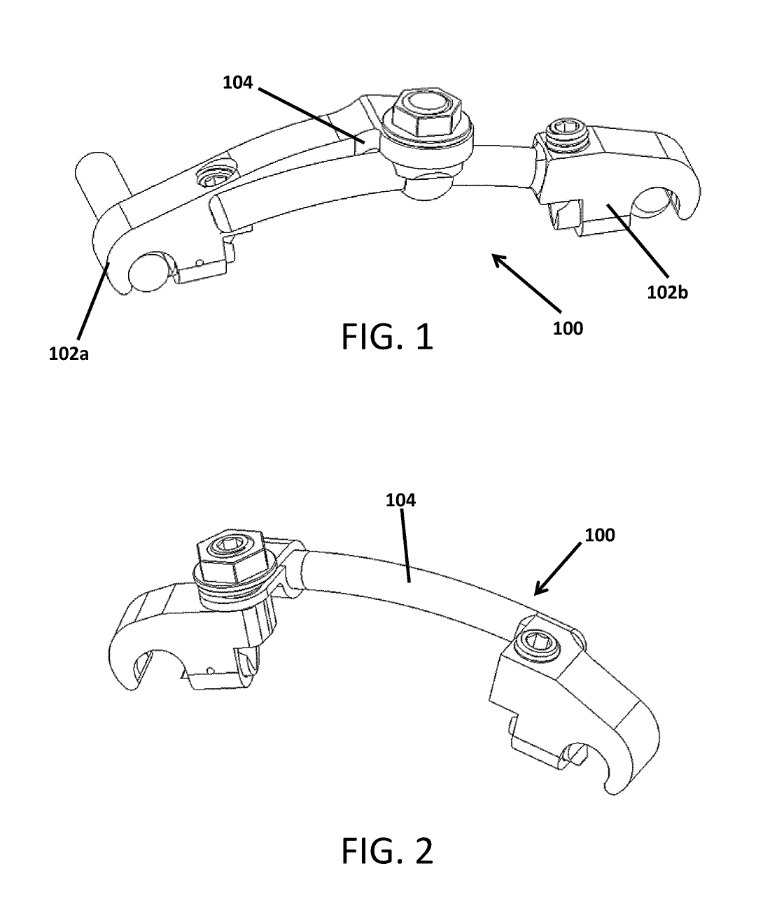

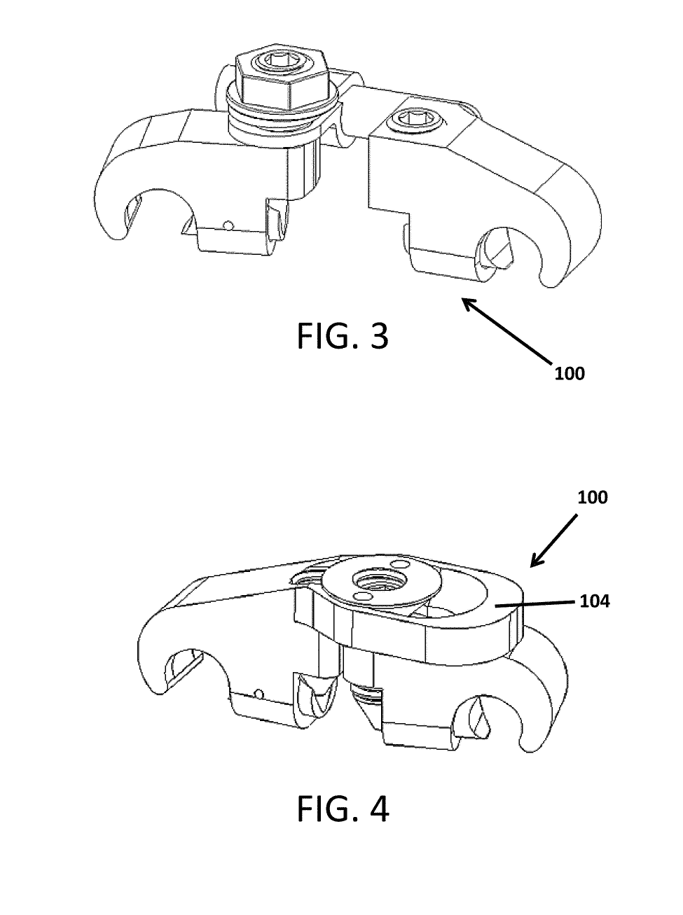

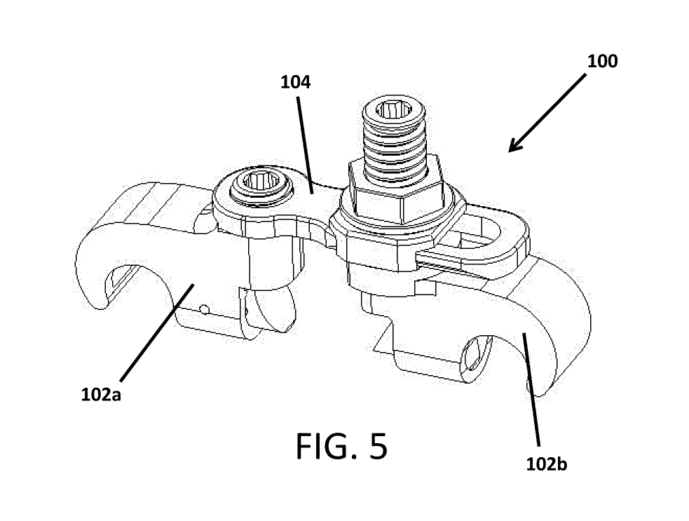

FIGS. 1-5 depict both long and short configurations of cross-connectors 100 that can be adapted to the invention according to certain embodiments. The cross-connectors 100 generally comprise a first clamping mechanism 102a disposed on a first end of a bridge member 104 and a second clamping mechanism disposed on an opposing second end 102b of the bridge member 104. The bridge member can be configured in various ways such as those depicted throughout FIGS. 1-5. As can be appreciated from the figures, the distance between the respective clamping mechanisms 102a, 102b can be adjusted in certain embodiments. Also, one or both of the clamping mechanisms 102a, 102b can be rotated with respect to the plane of the bridge member 104.

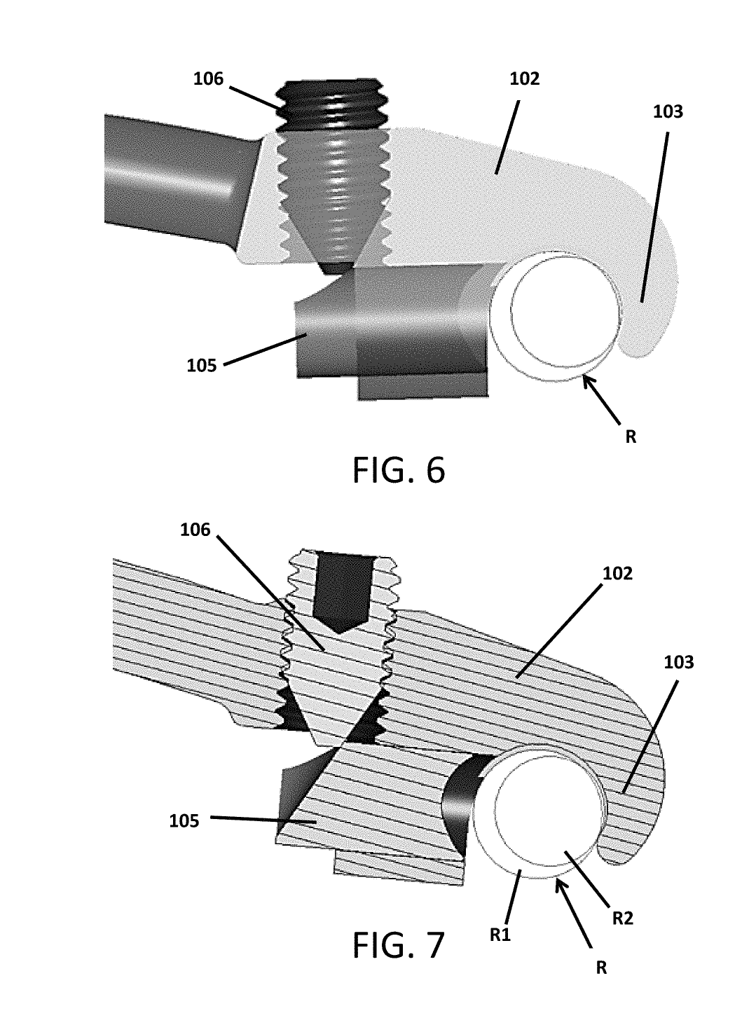

Referring now to FIGS. 6-11, details of the clamping mechanisms 102a, 102b (hereinafter generally designated with reference number 102) will now be discussed.

The clamping mechanism 102 includes a body, which defines a fixed hook portion 103, and a cross-slide cam 105 disposed in the body. A forward end of the cross-slide cam moves toward and away from the fixed hook portion. A tapered set screw 106 disposed in the body and oriented 90 degrees to the travel of the cross-slide cam 105. The set screw drives the cross-slide cam toward the fixed hook portion as the set screw is moved downward through the body of the clamping mechanism 102.

The forward end of the cross-slide cam 105 defines a curved surface that can be a constant radius, or the radius can be complex such that it defines two different radiused portions, or the radius can define another suitable type of curve to conform to a range of rod diameters. The back or rear end of the cross-slide cam 105 defines a planar surface portion that is angled with respect to the vertical plane. The side surface of the cross-slide cam 105 can be smooth, or it can define registration features such as recessed grooves or raised protrusions to maintain rotational alignment with respect to the fixed hook portion. The respective cam sliding surface of the fixed hook portion would receive complimentary protrusions or grooves to facilitate the registration.

Fixed hook portion 102 defines a curved inner surface that can be a constant radius, or the radius can be complex such that it defines two different radiused portions, or the radius can define another suitable type of curve to conform to a range of rod diameters.

The cross-slide cam 105 can be withdrawn rearwardly sufficient to provide clearance to insert the rod R between the forward end of the cross-slide cam 105 and the inner surface of the hook portion 102.

When the set screw 106 is fully withdrawn or retracted as shown in FIGS. 6-7, the cross-slide cam 105 moves rearwardly away from the fixed hook 103, thereby allowing the rod R to have sufficient clearance to be installed and seated into the opening of the fixed hook 103.

Note that two different rod diameters R1 and R2 are shown in FIGS. 6-11 for illustration and explanation purposes.

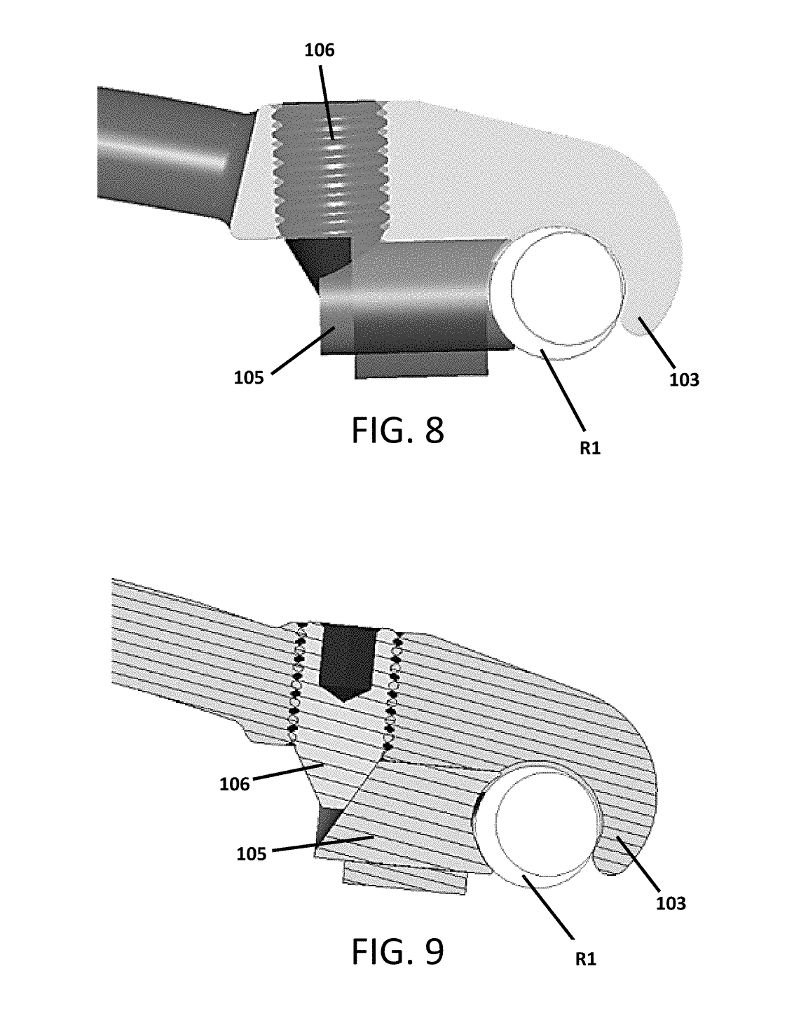

When the set screw 106 is driven down against the rear surface of the cross-slide cam 105, the radius of the front end of the cross-slide cam can engage rod R1 (whose diameter is larger than that of the second rod R2) as shown in FIGS. 8-9. This movement simultaneously presses the rod R1 against the inner surface of the fixed hook 103.

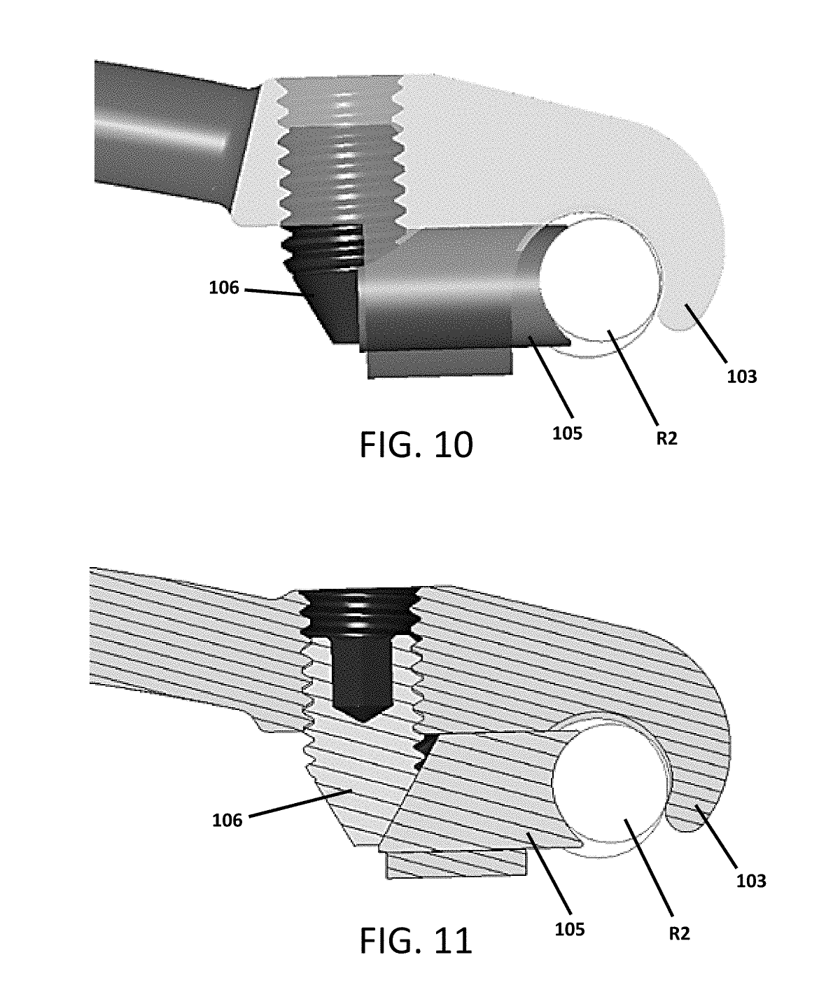

Further downward movement of the set screw 106 against the rear of the cross-slide cam 105 will bring the curved forward surface of the cross-slide cam 105 into engagement with the second rod R2 (whose diameter is smaller than that of the first rod R1) as shown in FIGS. 10-11. This movement simultaneously presses the rod R2 against the inner surface of the fixed hook 103. Thus, the same cross-connector can be used to secure either or both rod diameters.

The set screw 106 can be adjusted to any setting between a fully-retracted position and a fully-extended (maximum travel) position of the cross-slide cam 105.

The tapered set screw 106 includes a 60 degree forward tip angle (from the horizontal plane) to engage the back surface of the cross-slide cam 105 which includes a corresponding 30 degree angle (from the horizontal plane). The downward travel of the set screw 106 thus translates to the horizontal movement of the cross-slide cam 105 by engagement of the matched angles of the respective contacting surfaces. The matched angled surfaces noted above also magnify the downward force of the screw 106 into the cross-slide cam 105 to provide a significantly stronger clamping force than if the angles were reversed. The invention need not be limited to the specific angles noted herein.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An adjustable spinal rod cross-connector, comprising:
a bridge member;
a first clamping mechanism disposed on a first end of the bridge member; and
a second clamping mechanism disposed on an opposing second end of the bridge member,
wherein each of the first and second clamping mechanisms comprise:
a cross-slide cam, including a forward surface defining a curved portion and a rear surface defining an angled plane with respect to a horizontal plane; and
a set screw movable in a direction perpendicular to a long axis of the cross-slide cam and located such that the set screw engages the angled plane of the rear surface of the cross-slide cam.

2. The adjustable spinal rod cross-connector of claim 1, wherein the set screw defines a beveled tip with a bevel angle defined with respect to the horizontal plane.

3. The adjustable spinal rod cross-connector of claim 2, wherein the bevel angle is sixty degrees and the angled plane forms a thirty degree angle with respect to the horizontal plane.

4. The adjustable spinal rod cross-connector of claim 2, wherein the bevel angle is greater than an angle defined between the angled plane and the horizontal plane.

5. The adjustable spinal rod cross-connector of claim 1, wherein each of the first and second clamping mechanisms further comprise a fixed hook portion that defines a curved inner surface opposite the curved portion of the cross-slide cam.

6. The adjustable spinal rod cross-connector of claim 5, wherein the cross-slide cam defines sufficient travel between a fully extended position and a fully retracted position to engage spinal rods having diameters ranging from 4.5 mm to 6.0 mm.

7. The adjustable spinal rod cross-connector of claim 5, wherein the cross-slide cam defines sufficient travel between a fully extended position and a fully retracted position to engage spinal rods having diameters of both 4.75 mm and 5.50 mm.

8. The adjustable spinal rod cross-connector of claim 1, wherein the cross-slide cam defines sufficient travel between a fully extended position and a fully retracted position to engage spinal rods having diameters ranging from 4.5 mm to 6.0 mm.

9. The adjustable spinal rod cross-connector of claim 1, wherein the cross-slide cam defines sufficient travel between a fully extended position and a fully retracted position to engage spinal rods having diameters of both 4.75 mm and 5.50 mm.

10. An adjustable clamping mechanism for a spinal rod cross-connector, the adjustable clamping mechanism comprising:
a clamp body defining a fixed hook portion, the fixed hook portion defining a curved inner surface;
a cross-slide cam disposed in the clamp body, including a forward surface defining a curved portion and a rear surface defining an angled plane, wherein the cross-slide cam is movable along a long axis thereof through the body so that the forward surface moves towards and away from the curved inner surface of the fixed hook portion; and
a set screw disposed in the body and oriented 90 degrees to the long axis of the cross-slide cam such that the set screw engages the angled plane of the rear surface of the cross-slide cam.

11. The adjustable clamping mechanism of claim 10, wherein the set screw defines a beveled tip with a bevel angle defined with respect to the horizontal plane.

12. The adjustable clamping mechanism of claim 11, wherein the bevel angle is sixty degrees and the angled plane of the rear surface of the cross-slide cam forms a thirty degree angle with respect to the horizontal plane.

13. The adjustable clamping mechanism of claim 11, wherein the bevel angle is greater than an angle defined between the angled plane of the rear surface of the cross-slide cam and the horizontal plane.

14. The adjustable clamping mechanism of claim 13, wherein the cross-slide cam defines sufficient travel between a fully extended position and a fully retracted position to engage spinal rods having diameters of both 4.75 mm and 5.50 mm.

15. The adjustable clamping mechanism of claim 10, wherein the cross-slide cam defines sufficient travel between a fully extended position and a fully retracted position to engage spinal rods having diameters of both 4.75 mm and 5.50 mm.

16. The adjustable clamping mechanism of claim 10, wherein the cross-slide cam defines sufficient travel between a fully extended position and a fully retracted position to engage spinal rods having diameters ranging from 4.5 mm to 6.0 mm.

17. A method of securing a spinal rod with a clamping mechanism, the method comprising:
disposing a spinal rod in an adjustable clamping mechanism between a forward end of a cross-slide cam and a curved inner surface of a fixed hook portion of the clamping mechanism;
turning a screw to move the screw into the adjustable clamping mechanism to move the cross-slide cam in a direction perpendicular to a direction of travel of the screw;
engaging a curved forward end of the cross-slide cam with the spinal rod to secure the spinal rod in the adjustable clamping mechanism between the curved forward end and the curved inner surface of the fixed hook portion.

18. The method of claim 17, further comprising deflecting an angled rear surface of the cross-slide cam with a beveled tip of the screw.

19. The method of claim 18, wherein an angle of the beveled tip with respect to a horizontal plane is greater than an angle of the angled rear surface of the cross-slide cam with respect to the horizontal plane.

20. The method of claim 17, further comprising defining a fully extended position of the cross-slide cam and a fully retracted position of the cross-slide cam to permit engagement with spinal rods having diameters of both 4.75 mm and 5.50 mm.
